(19) United States
(12) Patent Application Publication
(10) Pub. No.: US 2007/0099852 A1
(43) Pub. Date: May 3, 2007

(54) CUCURBITACIN COMPOUNDS

(75) Inventors: Fathi T. Halaweish, Brookings, SD (US); Judit Bartalis, Chester, VA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SOUTH DAKOTA STATE UNIVERSITY, Brookings, SD (US)

(21) Appl. No.: 11/469,145

(22) Filed: Aug. 31, 2006

Related U.S. Application Data
(60) Provisional application No. 60/713,181, filed on Aug. 31, 2005.

(51) Int. Cl. C07J 17/00; C07J 9/00; A61K 31/704; A61K 31/573

(52) U.S. Cl. 514/26; 514/169; 536/6.1; 552/540

(57) ABSTRACT

Cucurbitacins, cucurbitacin derivatives, and methods for making and using the same are provided.

### FIELD OF THE INVENTION

The present invention pertains to cucurbitacin compounds, compositions, and methods for synthesizing and using cucurbitacin compounds. More particularly, the present invention pertains to cucurbitacin compounds that have anti-proliferative and/or hepatoprotective properties and the synthesis of cucurbitacin compounds.

### BACKGROUND

Cucurbitacins are highly oxygenated tetracyclic triterpenes that are produced in plants of the family Cucurbitaceae (e.g., gourds, zucchini, cucumber, melons, pumpkins, squash, etc.). Because of their bitterness, cucurbitacins work as a natural defense mechanism in these plants against phytophagous animals. Cucurbitacins also have a strong feeding stimulant effect on Diabroticina (e.g., cucumber beetles). Because of this phenomenon, pest control agents have been developed that combine cucurbitacins with insecticides to control several species of diabroticites.

### BRIEF SUMMARY

A number of alternative cucurbitacin compounds, methods for making cucurbitacin compounds, and uses for cucurbitacin compounds are disclosed. At least some of these compounds have a number of desirable properties such as anti-proliferative and/or hepatoprotective properties. These properties may be useful in the treatment of a number of human diseases such as cancer and liver fibrosis as well as protect the liver against viral infection and toxins.

The above summary of some embodiments is not intended to describe each disclosed embodiment or every implementation of the present invention. The Figures, Detailed Description, and Examples, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a chromatogram depicting cucurbitacins separated via HPCL (Cucurbitacins HPCL separation on Alltima C18 (250 mm×4.6 mm, 5 μm). Conditions: 30-70% ACN in water in 57 min, flow rate 1 ml/min);

FIG. 2 is another chromatogram depicting cucurbitacins separated via HPCL (Cucurbitacins HPCL separation on Alltima C18 (250 mm×4.6 mm, 5 μm). Conditions: 60-75% MeOH in water in 50 min, flow rate 1 ml/min);

FIG. 3 is a graph depicting the relationship between toxicity and chromatographic hydrophobicity index for cucurbitacins (Relationship between cucurbitacins toxicity on HepG2 cells and CHI measured in acetonitrile (a, where y=−0.0308x+3.53, r=0.901) or in methanol (b, where y=−0.0955x+7.7677, r=0.918));

FIG. 4 is a graph depicting the effect of silybin on HepG2 cells in presence or absence of CCl₄

FIG. 5 is a graph depicting cucurbitacin cytoprotection against CCl₄ toxicity on HepG2 cells at 20% and 50% of their IC₅₀ concentration;

FIG. 6 depicts normal, healthy HSC-T6 and HepG2 cells; A) HSC-T6 cells at day 1 at 20× and 40× magnification, respectively, B) HepG2 cells;

FIG. 7 depicts the cell morphology at different time intervals for positive (with serum) and negative (no serum) controls and for cucurbitacin E glucoside at 150 μM (Experiment 1), PC=positive control, NC=negative control; and

FIG. 8 is a graph depicting cucurbitacin cytotoxicity and activity on HSC-T6 proliferated in serum.

### DETAILED DESCRIPTION

The following description should be read with reference to the drawings wherein like reference numerals indicate like elements throughout the several views. The detailed description and drawings illustrate example embodiments of the claimed invention.

Definitions

All scientific and technical terms used in this application have meanings commonly used in the art unless otherwise specified. As used in this application, the following words or phrases have the meanings specified.

For the following defined terms, these definitions shall be applied, unless a different definition is given in the claims or elsewhere in this specification.

The terms “cucurbitacin”, “cucurbitacin compound”, and “cucurbitacin derivative” are used interchangeably in the detailed description to mean compounds having any of the chemical formulas listed below.

As used herein, the term “alkyl” refers to a straight or branched chain monovalent hydrocarbon radical having a specified number of carbon atoms. Alkyl groups include those with one to twenty carbon atoms. Alkyl groups may be unsubstituted or substituted with those substituents that do not interfere with the specified function of the composition. Substituents include alkoxy, hydroxy, mercapto, amino, alkyl substituted amino, or halo, for example. Examples of “alkyl” as used herein include, but are not limited to, methyl, ethyl, n-propyl, n-butyl, n-pentyl, isobutyl, and isopropyl, and the like.

As used herein, the term “alkene” refers to a straight or branched chain hydrocarbon having one or more carbon-carbon double bonds. Alkene groups typically include those with two to twenty carbon atoms. Examples of “alkene” as used herein include, but are not limited to, ethene, propene, 3-methyl-1-butene, 2-methylpropene, 2-methyl-1,3-butadiene, (E)-3-methyl-3-hexene, and the like.

As used herein, “acyl” refers to a carbonyl group with an alkyl group attached. Acyl groups typically may contain one to about 20 carbon atoms. Some examples include methanoyl (formyl), ethanoyl (acetyl), propanoyl, benzoyl, etc.

As used herein, “carboxylic acid” refers to compounds that contain the carbonyl functional group RCOOH. Carboxylic acids typically may contain one to about 20 carbon atoms. Some examples include methanoic acid, ethanoic acid, propanoic acid, butanoic acid, etheanedioic acid, propanedioic acid, butanedioic acid, benzenecarboxylic acid, and the like.

As used herein, “alkoxide” (or “alkoxide anion”) are alcohols where hydroxyl proton is removed (e.g., via reduction) to define an —O-alkyl group wherein alkyl is as defined above. Some examples include methoxide, ethoxide, propoxide, isopropoxide, etc.

As used herein, the term “hydroxide” refers to the substituent —OH and may be used interchangeably therewith.

As used herein, the term “halogen” or “halo” shall include iodine, bromine, chlorine and fluorine.

As used herein, the term “sugar” refers to carbohydrates including monosaccharides, disaccharides, oligosaccharides, and polysaccharides having, for example, four (tetrose), five (pentose), six (hexose), seven (heptose), or more carbon atoms. Some examples of monosaccharides sugars include allose, altrose, glucose, mannose, gulose, idose, galactose, talose, ribose, arabinose, xylose, lyxose, erthrose, threose, and glyceraldehyde. Some examples of disaccharides include cellobiose, maltose, lactose, gentiobiose, and sucrose. Some examples of oligosaccharides and/or polysaccharides include cellulose, starch, amylase, amylase, amylopectin, and glycogen. The sugar may be an aldose sugar (i.e., a sugar having an aldehyde functional group) or a ketose sugar (i.e., a sugar having a ketone functional group). The sugar may be a reducing sugar (i.e., a sugar oxidized by Tollens' reagent, Benedict's reagent, or Fehling's reagent) or a non-reducing sugar (i.e., a sugar not oxizided by Tollens' reagent, Benedict's reagent, or Fehling's reagent). The sugar may be cyclic (e.g., furanose, pyranose, etc.) or non-cyclic. The sugar may be either the D or L enantiomer, may rotate polarized light in either the (+) or the (−) direction, and may be either the α or β anomer.

As used herein, “pharmaceutically acceptable carrier” means any material which, when combined with the compound of the invention, allows the compound to retain biological activity, such as anti-proliferative and/or hepatoprotective activity. Examples include, but are not limited to, any of the standard pharmaceutical carriers such as a phosphate buffered saline solution, water, emulsions such as oil/water emulsions, and various types of wetting agents. Compositions comprising such carriers are formulated by conventional methods.

Compounds of the Invention

Compounds of the invention include cucurbitacins having the formula:

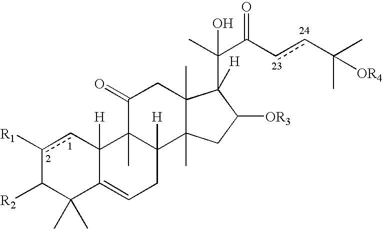

where:

R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo.

R₂ is —H, —OH, ═O, or (C₁-C₁₂)carboxylic acid.

R₃ is —H, (C₁-C₁₂)acyl, (C₁-C₁₂)alkyl, (C₁-C₁₂)alkene, or (C₁-C₁₂)carboxylic acid.

R₄ is —H, (C₁-C₁₂)acyl, (C₁-C₁₂)alkyl, (C₁-C₁₂)alkene, or (C₁-C₁₂)carboxylic acid.

Carbon 1 and carbon 2 may be bonded with a single bond or a double bond.

Carbon 23 and carbon 24 may be bonded with a single bond or a double bond.

Compounds of the invention also include cucurbitacins having the formula:

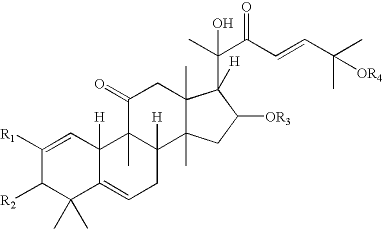

where:

R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo.

R₂ is —H, —OH, ═O, or (C₁-C₁₂)carboxylic acid.

R₃ is —H, (C₁-C₁₂)acyl, (C₁-C₁₂)alkyl, (C₁-C₁₂)alkene, or (C₁-C₁₂)carboxylic acid.

R₄ is —H, (C₁-C₁₂)acyl, (C₁-C₁₂)alkyl, (C₁-C₁₂)alkene, or (C₁-C₁₂)carboxylic acid.

Compounds of the invention also include cucurbitacins having the formula:

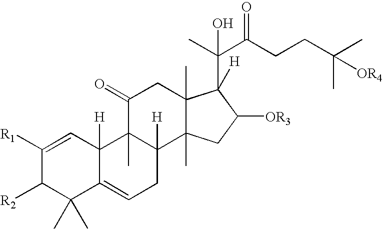

where:

R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo.

R₂ is —H, —OH, ═O, or (C₁-C₁₂)carboxylic acid.

R₃ is —H, (C₁-C₁₂)acyl, (C₁-C₁₂)alkyl, (C₁-C₁₂)alkene, or (C₁-C₁₂)carboxylic acid.

R₄ is —H, (C₁-C₁₂)acyl, (C₁-C₁₂)alkyl, (C₁-C₁₂)alkene, or (C₁-C₁₂)carboxylic acid.

Compounds of the invention also include cucurbitacins having the formula:

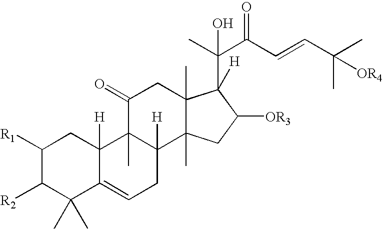

where:

R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo.

R₂ is —H, —OH, ═O, or (C₁-C₁₂)carboxylic acid.

R₃ is —H, (C₁-C₁₂)acyl, (C₁-C₁₂)alkyl, (C₁-C₁₂)alkene, or (C₁-C₁₂)carboxylic acid.

R₄ is —H, (C₁-C₁₂)acyl, (C₁-C₁₂)alkyl, (C₁-C₁₂)alkene, or (C₁-C₁₂)carboxylic acid.

Compounds of the invention also include cucurbitacins having the formula:

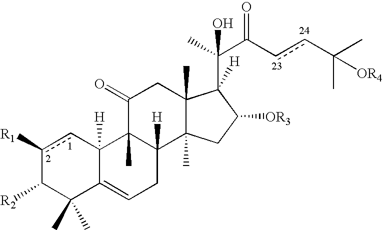

where:

R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo.

R₂ is —H, —OH, ═O, or (C₁-C₁₂)carboxylic acid.

R₃ is —H, (C₁-C₁₂)acyl, (C₁-C₁₂)alkyl, (C₁-C₁₂)alkene, or (C₁-C₁₂)carboxylic acid.

R₄ is —H, (C₁-C₁₂)acyl, (C₁-C₁₂)alkyl, (C₁-C₁₂)alkene, or (C₁-C₁₂)carboxylic acid.

Carbon 1 and carbon 2 may be bonded with a single bond or a double bond.

Carbon 23 and carbon 24 may be bonded with a single bond or a double bond.

Compounds of the invention also include cucurbitacins having the formula:

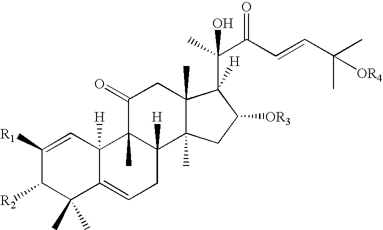

where:

R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo.

R₂ is —H, —OH, ═O, or (C₁-C₁₂)carboxylic acid.

R₃ is —H, (C₁-C₁₂)acyl, (C₁-C₁₂)alkyl, (C₁-C₁₂)alkene, or (C₁-C₁₂)carboxylic acid.

R₄ is —H, (C₁-C₁₂)acyl, (C₁-C₁₂)alkyl, (C₁-C₁₂)alkene, or (C₁-C₁₂)carboxylic acid.

Compounds of the invention also include cucurbitacins having the formula:

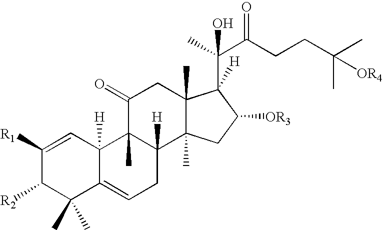

where:

R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo.

R₂ is —H, —OH, ═O, or (C₁-C₁₂)carboxylic acid.

R₃ is —H, (C₁-C₁₂)acyl, (C₁-C₁₂)alkyl, (C₁-C₁₂)alkene, or (C₁-C₁₂)carboxylic acid.

R₄ is —H, (C₁-C₁₂)acyl, (C₁-C₁₂)alkyl, (C₁-C₁₂)alkene, or (C₁-C₁₂)carboxylic acid.

Compounds of the invention also include cucurbitacins having the formula:

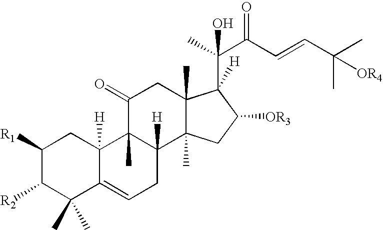

where:

R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo.

R₂ is —H, —OH, ═O, or (C₁-C₁₂)carboxylic acid.

R₃ is —H, (C₁-C₁₂)acyl, (C₁-C₁₂)alkyl, (C₁-C₁₂)alkene, or (C₁-C₁₂)carboxylic acid.

R₄ is —H, (C₁-C₁₂)acyl, (C₁-C₁₂)alkyl, (C₁-C₁₂)alkene, or (C₁-C₁₂)carboxylic acid.

Cucurbitacins and uses for Cucurbitacin Compounds

Cancer and/or anti-proliferative drugs are often associated with a number of side effects. Cytotoxicity is among some of these side effects. In some cases, cytotoxicity is among the desired effects for anti-proliferative drugs because killing cancerous cells is often the goal of a cancer intervention. However, because some cancer drugs cannot discriminate between “healthy” cells and cancerous cells, there is an ongoing need for improved cancer and/or anti-proliferative drugs that selectively target cancerous cells and/or specific target regions while having fewer side effects or less cytotoxicity at other targets.

Because the liver is often involved in the metabolism of many drugs, often the liver is a major site of cytotoxicity of drugs. For example, a cancer drug may be used to treat a cancer in another region of the body (e.g., prostate, brain, ovary, etc.) and still exhibit a cytotoxic effect on the liver. This may result in liver injury such as necrosis, cholestasis or steatosis. There is an ongoing need for improved drugs that are less toxic to the liver and/or possess hepatoprotective effects and/or properties. Moreover, inflammatory reactions are also triggered in many liver diseases, for example, as the consequence of the introduction of a toxin, drug, or infectious agent. These reactions can induce a repair process to restore the original functions of the hepatic tissue. The failure to eliminate the noxious agent, in addition to the disruption of regulatory mechanisms may lead to the development of chronic liver inflammation. Thus, a further need exists for drugs that protect the liver.

The compounds of the present invention, shown above, are derivatives or analogs of cucurbitacins that have anti-proliferative properties and/or hepatoprotective properties. Because of this, these compounds may be useful for the treatment of a number of mammalian diseases or conditions such as cancer, liver disease, liver failure, cirrhosis, combinations thereof, and the like. Accordingly, in some embodiments cucurbitacins of the formula above (and/or pharmaceutically acceptable salts thereof) can be combined with a pharmaceutically acceptable carrier and administered to a patient to treat cancer, liver disease, liver failure, cirrhosis, combinations thereof, and the like. The cancer may include cancer of any body tissue. For example, the cancer may include prostate, brain, or ovarian cancer.

The compounds of the present invention can be formulated as pharmaceutical compositions (hereafter “compositions”, which include one or more of the compounds described above, the pharmaceutically acceptable salts thereof, one or more of the compounds described above combined with a pharmaceutically acceptable carrier, or combinations thereof) and administered to a mammalian host, such as a human patient in a variety of forms adapted to the chosen route of administration, i.e., orally or parenterally, by intravenous, intramuscular, topical or subcutaneous routes.

Thus, the compositions may be systemically administered, e.g., orally, in combination with a pharmaceutically acceptable carrier (e.g., such as those listed above and/or an inert diluent or an assimilable edible carrier). They may be enclosed in hard or soft shell gelatin capsules, may be compressed into tablets, or may be incorporated directly with the food of the patient's diet. For oral therapeutic administration, one or more of the compounds may be combined with one or more excipients and used in the form of ingestible tablets, buccal tablets, troches, capsules, elixirs, suspensions, syrups, wafers, and the like. Such compositions and preparations may contain at least 0.1% of the one or more compounds, for example. The percentage of the one or more compounds in a given composition and preparations may, of course, be varied and may conveniently be between about 2 to about 60% of the weight of a given unit dosage form. The amount of the example one or more compounds in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For example, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the compositions may be incorporated into sustained-release preparations and devices.

The compositions may also be administered intravenously or intraperitoneally by infusion or injection. Solutions that include one or more of the compounds can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations may contain a preservative to prevent the growth of microorganisms.

The pharmaceutical dosage forms suitable for injection or infusion can include sterile aqueous solutions or dispersions or sterile powders comprising one or more of the compounds, which are adapted for the extemporaneous preparation of sterile injectable or infusible solutions or dispersions, optionally encapsulated in liposomes. In at least some embodiments, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compounds in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the compositions may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the compositions can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the above compositions can be determined by comparing their in vitro activity, and in vivo activity in animal models. Methods for the extrapolation of effective dosages in mice, and other animals, to humans are known to the art. The compounds may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### EXAMPLES

The invention may be further clarified by reference to the following Examples, which serve to exemplify some of the preferred embodiments, and not to limit the invention in any way.

### Example 1

Plants secondary metabolites represent tremendous resources for scientific and clinical researches as well as for new drug development. Cucurbitacins are known in folk medicine for their strong purgative, anti-inflammatory, and hepatoprotective activities. However, the biological activity of cucurbitacins often occurs at doses that are close to their toxic dose.

Lipophilicity is one of the major factors that influences the transport, absorption, and distribution of chemicals in biological systems, and it is a predominant descriptor of the pharmacodynamic, pharmacokinetic and toxic aspects of drug activities in quantitative structure-activity relationship (QSAR) studies. In the 1960s, Hansch's octanol-water partition coefficient P_{oct} (P_{oct}=C_{oct}/C_{water}; C: analyte concentration) became the standard parameter to measure lipophilicity for both experimental and theoretical investigations (Hansch and Leo, Fundamentals and Applications in Chemistry and Biology, American Chemical Society, Washington D.C., 1995, the entire disclosure of which is herein incorporated by reference). The octanol-water partition coefficients can be obtained from other solvent systems, with certain restrictions, by applying Collander's equation (Collander, Acta Chem. Scand. 5 (1951) 774, the entire disclosure of which is herein incorporated by reference): log P₁=a log P₂+b. Reverse-phase high pressure liquid chromatography (RP-HPLC) has been long recognized as a potential method for lipophilicity determination, where mainly hydrophobic forces dominate the retention process. Moreover, the mobile phase/stationary phase interface models better the biological partitioning processes than the solute partitioning in the bulk octanol/water phase. The chromatographic retention data is a linear free-energy related parameter and it is a more reliable descriptor in QSAR than the estimated or calculated hydrophobic, electronic and/or steric parameters. Chromatographic hydrophobicity index (CHI) is deduced from the retention data and reflects not only the lipophilicity of the compound but it approximates the concentration of organic phase required achieving an equal distribution of analyte between the mobile phase and stationary phase. Thus, hydrophobicity index is a useful tool in method development.

One of the goals for drug development of cucurbitacins is to develop analogues with enhanced or typical biological activity and reduced toxicity. For this study, a human HepG2 cell line was chosen for its ability to predict basal human cytotoxicity.

This work presents a precise and reliable technique to study the effect of structural modification on cucurbitacins cytotoxicity. The basal cytotoxicity of seventeen cucurbitacin analogues (Table 1, which lists compounds 1-18, please note that compound 10 was not included in the cytotoxicity assay) was monitored on HepG2 cells, and their hydrophobicity was calculated in different ways. The lipophilic parameters are the CHI, measured by RP-HPLC, and log P and C log P estimated with ALOGPS software (Virtual Computational Chemistry Laboratory, www.vcclab.org). In order to have a larger number of compounds, some cucurbitacins were isolated from plants and others generated by alkylation and acetylation of enolic analogues. Cucurbitacins drug development may seek derivatives with low cytotoxicity, and correlation of lipophilicity with in vitro toxicity may lead to important conclusions regarding this issue.

Ripe fruits of Cucurbita texana (Cucurbitaceae) were received from Dr. D. W. Tallamy (University of Delaware, Newark, Del.). The fruits were cut and homogenized with methanol (MeOH), filtered, and the solvent removed under reduced pressure. The residue was subjected to flash column chromatography (silica gel G60) with gradient elution (hexane/ethyl acetate and then ethyl acetate/MeOH of increasing polarity) and the fractions were screened using NP-TLC (silica gel, UV₂₅₄, 250 μm layer). TLC plates were developed with toluene:ethyl acetate 40:60 solvent mixture, and visualized for the Δ^{23,24} cucurbitacins (see Table 1) with vanillin/orthophosphoric acid or for the diosphenols with FeCl₃ solution. Fractions were further separated using preparative NP-TLC (silica gel, UV₂₅₄, 2 mm layer) under similar developing conditions to the analytical TLC and bands were visualized with UV light. Cucurbitacins ¹³C and ¹H NMR spectra (Bruker 400 MHz) were recorded in CDCl₃ and compared to published data. Additional amounts of cucurbitacin glycosides were isolated by preparative HPLC from the concentrate of Citrullus lanatus (Cucurbitaceae) (Florida Food Products, Eustis, Fla.).

TABLE 1
Cucurbitacins used for this assay.

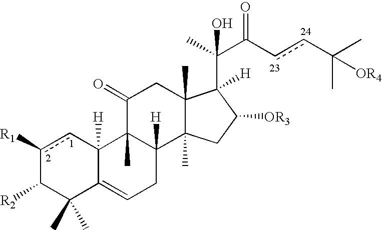

| Compound | | | | | | |
|---|---|---|---|---|---|---|
| No. | Cucurbitacin | R₁^{d} | R₂^{e} | R₃ | R₄ | Other |
| 1 | I GIuc^{a,b} | Glu | ═O | H | H | Δ^{1,2}, Δ^{23,24} |
| 2 | E Gluc^{a,b} | Glu | ═O | H | Ac | Δ^{1,2}, Δ^{23,24} |
| 3 | D^{b} | OH | ═O | H | H | Δ^{23,24} |
| 4 | iso-D^{b} | ═O | OH | H | H | Δ^{23,24} |
| 5 | I^{b} | OH | ═O | H | f-I | Δ^{1,2}, Δ^{23,24} |
| 6 | I-Me^{c} | O-Me | ═O | H | H | Δ^{1,2}, Δ^{23,24} |
| 7 | L-Me^{c} | O-Me | ═O | H | H | Δ^{1,2} |
| 8 | I-Et^{c} | O-Et | ═O | H | H | Δ^{1,2}, Δ^{23,24} |
| 9 | B^{b} | OH | ═O | H | Ac | Δ^{23,24} |
| 10 | iso-B^{b} | ═O | OH | H | Ac | Δ^{23,24} |
| 11 | I-iPr^{c} | O-iPr | ═O | H | H | Δ^{1,2}, Δ^{23,24} |
| 12 | I-nPr^{c} | O-riPr | ═O | H | H | Δ^{1,2}, Δ^{23,24} |
| 13 | E^{b} | OH | ═O | H | Ac | Δ^{1,2}, Δ^{23,24} |
| 14 | E-Me^{c} | O-Me | ═O | H | Ac | Δ^{1,2}, Δ^{23,24} |
| 15 | E-Et^{c} | O-Et | ═O | H | Ac | Δ^{1,2}, Δ^{23,24} |
| 16 | E-iPr^{c} | O-iPr | ═O | H | Ac | Δ^{1,2}, Δ^{23,24} |
| 17 | E-Me-Ac^{c} | O-Me | ═O | Ac | Ac | Δ^{1,2}, Δ^{23,24} |
| 18 | E-nPr^{c} | O-nPr | ═O | H | Ac | Δ^{1,2}, Δ^{23,24} |

^{a}β-D-glucopyranose;
^{b}isolated from plants;
^{c}generated by semi-synthesis;
^{d}OH is positioned in β;
^{e}OH is positioned in α

HPLC Separation

We used Dynamax liquid chromatograph (Varian Chromatography Systems) with PDA-2 photodiode array UV detector, controlled by the Dynamax PC Chromatography Data System (v. 1.9) software and Dynamax dual pump solvent delivery system, model SD-200. Cucurbitacins final purification and separation was conducted on Econosil C18 (Alltech; 250 mm×22 mm, 10 μm) preparative column at flow rate of 13.00 ml/min, and at gradient elution in acetonitrile (Pharmco, Brookfield, Conn.; 20-55% in 50 min), or MeOH (Pharmco; 60-75% in 50 min). Cucurbitacins analytical separation was optimized on Alltima C18 (Alltech; 250 mm×4.6 mm, 5 μm) HPLC column, at gradient elution in acetonitrile (ACN, 30-70% ACN in 57 min), and in MeOH (60-75% MeOH in 50 min). Cucurbitacins stock concentration of 10-2M in DMSO:ethanol (1:1) was standardized against pure cucurbitacin I (Indofine Chemical Company, Hillsborough, N.J.) by analytical HPLC means. Compounds CHI was measured in both ACN, by using Alltima C18 column, and in MeOH, by using Econosil C18 column (Alltech; 150 mm×4.6 mm, 5 μm). Analytical separations were conducted at a flow rate of 1 ml/min. The aqueous phase was buffered for the CHI measurement. For this purpose, solid ammonium acetate (Fisher Sci. Co., Fair Lawn, N.J.) was dissolved in deionized distilled water at 50 mM final concentration and its pH adjusted to 7.0.

Chromato Graphic Hydrophobicity Index

CHI Measurement in ACN

All standard compounds were purchased from Acros (Acros Organics, NJ). The chromatographic lipophilicity or hydrophobicity was determined applying Valkó's technique (Valkó et al., Anal. Chem. 69 (1997) 2022, the entire disclosure of which is herein incorporated by reference). A standard mixture of seven compounds was prepared in solution: theophylline (compound 19), benzimidazole (compound 20), acetophenone (compound 21), indole (compound 22), propiophenone (compound 23), butyrophenone (compound 24), and valerophenone (compound 25). In the first approach, the mixture of compounds 19-25, dissolved in water:ACN (1:1), was injected at isocratic elution of 40, 45, 50, 55, and 60% ACN. The retention factor, log k=log((t_{R}-t₀)/t₀), was calculated for each analyte from five good injections of 10 μm sample. The dead time (t₀) was measured by injecting NaNO₃ together with the sample. Then, the log k values were plotted against isocratic ACN concentrations to establish the linear regression equations for each analyte. From each straight line the isocratic hydrophobicity index was computed, φ₀=(intercept/slope). Further, the calibration mixture was injected at fast gradient elution, 0-22 min 0-100% ACN, and three additional minutes at 100% ACN. The φ₀ values for the test compounds were plotted against gradient retention time and the linear equation determined from the following equation:
φ₀=CHI=At_{R}+B (1)

A mixture of 18 cucurbitacins analogues (Table 1) was injected under similar gradient elution and, from each peak's retention time, the CHI values were deduced applying Eq. (1). In the second approach, Eq. (1) was generated from the correlation between the published CHI values and the fast gradient elution of compounds 19-25, colchicine (compound 26), and phenyltheophylline (compound 27). The gradient elution conditions were similar to the one from the first approach.

CHI Measurement in MeOH

A standard mixture of 10 compounds including 19-21, 23-27, aniline (compound 28) and bromobenzene (compound 29), dissolved in MeOH, was injected at five isochratic elution, at 40, 45, 50, 55, and 60% MeOH. Then the mixture was injected at fast gradient elution to establish the correlation from Eq. (1). The fast linear gradient elution was optimized for 30-100% MeOH in aqueous buffer with 10 min runtime.

Structural Modification

Alkylation

The C2 hydroxyl of enolic analogues, such as cucurbitacins E cucurbitacins I, was alkylated by the Williamson ether synthesis. Pure cucurbitacins (2 mg) and freshly dried anhydrous K₂CO₃ (3 g) were mixed and refluxed in acetone under N₂ with continuous stirring for 3 days. During this period, two portions of alkyl iodide, or RI (R: Me-, Et-, iPr-, or nPr-; 50 ml) were added at 24 h intervals. The solution was filtered and the salt washed twice with acetone. The combined filtrate and washings was evaporated under air and the residue further purified by preparative RP-HPLC.

Acetylation

Cucurbitacin E-Me ether (2 mg) was acetylated at C16 position overnight at room temperature in dry pyridine (5 ml) and acetic anhydride (5 ml). The mixture was decomposed with cold water and the product extracted in methylene chloride, then evaporated and further purified by preparative RP-HPLC.

Enzymatic Hydrolysis

Additional amount of aglycons were generated by the enzymatic hydrolysis of saponins cucurbitacin E β-glucoside and I β-glucoside, using β-glucosidase enzyme (Worthington, Lakewood, N.J.). A ratio of 1:4 saponin to enzyme was suspended in acetate buffer at pH 5 and stirred continuously under N₂ for 3 days in a water bath, at 37° C. Half portion of enzyme was added to the mixture after 2 days of stirring.

Cell Culture and Induction of Toxicity

HepG2 (human hepatocellular carcinoma, ATCC) cells were grown in EMEM (Gibco, Grand Island, N.Y.) supplemented with 10% fetal bovine serum (FBS), and 1% penicillin/fungizone mixture (Gibco). Thabrew's optimized procedure (Thabrew et al., J. Pharm. Pharmcol. 49 (1994) 1442, the entire disclosure of which is herein incorporated by reference) was followed. Cells were batch cultured for 10 days, then seeded at concentration of 30,000 cells/well in fresh media in 96-well microtiter plastic plates at 37° C. for a day. Then cells were exposed to different concentrations of cucurbitacins at final volume of 100 μl/well. Five-fold serial dilution of compounds was carried out in the plate for five consecutive wells. After 24 h of incubation with chemicals, live cells were visualized using the MTT viability assay (Promega, Madison, Wis.). The absorbance was measured at 570 nm. Negative (without cells) and positive (without test chemicals) controls were also incubated with each plate. The endpoint was determined from the exponential curve of viability versus concentration as IC₅₀, which represents the concentration of compound that kills 50% of the cells. At least three reproducible experiments were performed per compound with three replicate wells per concentration.

Calculations

The estimated log P and C log P octanol/water partition coefficients for cucurbitacins were obtained by means of the on-line software ALOGPS v. 2.1 (Virtual Computational Chemistry Laboratory, www.vcclab.org). The log P calculation is based on the neural network ensemble analysis, where the molecular structure was represented by the electrotopological state indices and the number of hydrogen and non-hydrogen atoms. The C log P partition coefficient is based on the fragmentation principle developed by Leo et al. The CLOGP program version 4.0 uses improved C log P calculation theory and it is running under evaluation license of BioByte Corporation.

The data analysis was carried out using the Microsoft Excel® 2000 software package. The correlation coefficient “r”, F-test, and t-test were the basis for testing the significance of fitting quality. In addition, the S/O (i.e., the ratio of standard error and range of observation) was introduced as a specific fitting error. The statistical residual variance RV was considered in assessment of the prediction error. RV is the ratio of prediction sum of squares (PRESS) and the total number of data n, and PRESS is:

$$PRESS = {\sum }_{j = 1}^{n} {( {Obs}_{j} - {Pred}_{j} )}^{2} \qquad ( 2 )$$

where Obsⱼ and Predⱼ are the collected and predicted values. High quality models should give S/O and RV values close to zero.
Results and Discussion

Cucurbitacin analogues were isolated from C. texana and C. lanatus, and diosphenols 5 and 13 were further modified by alkylation and esterification (Table 1). The alkylation of compounds 5 and 13 and acetylation of 14 yielded 100% the product. On the other hand, methylation of a mixture containing non-separable cucurbitacins I and L generated only L-Me ether. The enzymatic hydrolysis of 1 and 2 yielded 35% of cucurbitacin 1 and 100% of cucurbitacin E, respectively; the transformation was not complete for 1 even though both 1 and 2 have β-glucosidic bond. Several attempts have been made to methylate the C2 hydroxyl of cucurbitacin B. Unfortunately, alkylation in the presence of a strong base (NaH, THF, RI, 50° C.) or reaction with diazomethane (freshly prepared CH₂N₂, HBF₄, CH₂Cl₂, 0° C.), destroyed the functional groups.

¹H and ¹³C NMR data of the isolated and modified cucurbitacins matched the published data. The new carbon shifts for the semi-synthesized compounds were identified, for the R₁ side chain: 55.0 ppm (CH₃—O) for compounds 6-8 and 17; 14.4 ppm (CH₃) and 63.4 ppm (CH₂—O) for 8 and 14; 21.5 ppm (CH₃) and 70.3 ppm (CH—O) for 11 and 16; 10.4 ppm (CH₃), 22.1 ppm (CH₂), and 69.4 ppm (CH₂—O) for 12 and 18. The R₃ group ¹³C-NMR shift of 17 was found at 19.9 ppm (CH₃) and 169.8 ppm (CO).

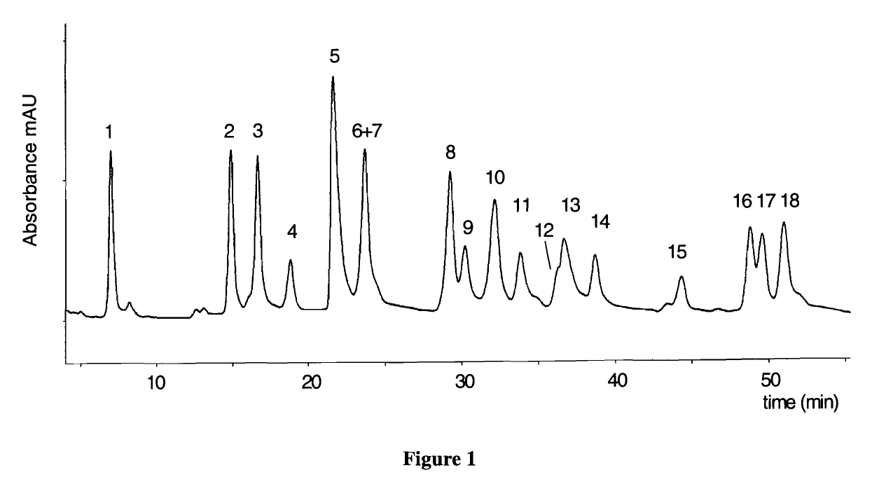

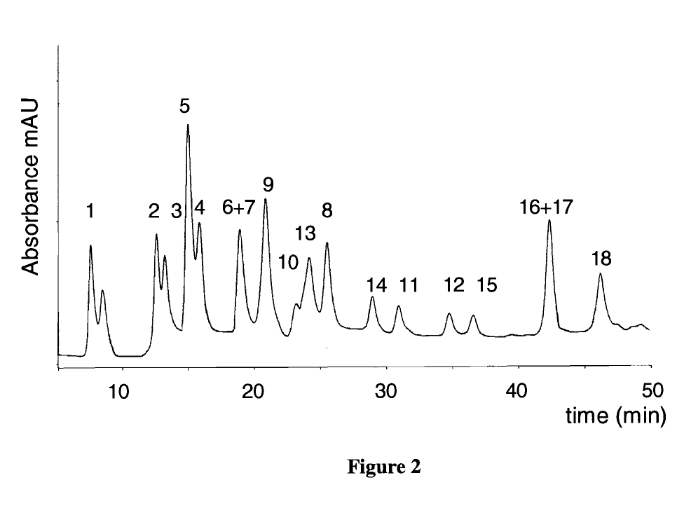

The RP-HPLC separation of cucurbitacin analogues was conducted in both aqueous ACN and MeOH. Chromatograms are illustrated in FIGS. 1 and 2, where peaks are numbered following the order in Table 1. Higher resolution was achieved in ACN than in MeOH organic phase. Interestingly enough, Alltima C18 HPLC column showed different selectivity toward the C25-OH derivatives 8, 11, and 12 in the two organic phase. Methanol is a good proton acceptor and tends to interact with hydroxylated molecules. This would suggest that compounds 8, 11, and 12, with an extra hydroxyl group relative to other derivatives, would elute faster in MeOH relative to ACN, contrary to what was actually happening. We can explain it with the fact that there are some complex interactions taking place between the solute and stationary phase. Abraham quantified these interactions (Abraham, Chem. Soc. Rev. 22 (1993) 73, the entire disclosure of which is herein incorporated by reference), and Valkó tailored Abraham's equation for various organic phases finding that both solute dipolarity and hydrogen-bond acidity had weaker influence over solute elution in methanol than in acetonitrile (Valkó et al., Curr. Med. Chem. 8 (2001) 1137, the entire disclosure of which is herein incorporated by reference).

Cucurbitacins lipophilicity was measured by RP-HPLC. The selectivity differences in the two organic phase prompted us to measure CHI in both ACN and MeOH organic phase. Due to its high viscosity, aqueous MeOH required a shorter column than the one applied for ACN. First, the C18 columns were calibrated against a standard mixture, and the relationships established between the fast gradient t_{R} and φ₀ or published CHI (see Eqs. (2)-(4) from Table 2). The CHI of the standard compounds is listed in Table 3. Second, cucurbitacins were injected at fast gradient elution under similar conditions, and their CHI calculated (Table 4) from Eqs. (2)-(4). Eqs. (2) and (4) involve the isochratic hydrophobicity index, φ₀, while Eq. (3) employs the earlier established gradient CHI in buffered ACN. Faster gradient elution did not improve statistically Eqs. (2)(4). The fitting quality and predictive power of Eq. (3) (CHI_{ACN2}) and Eq. (4) (CHI_{MeOH}) are relatively high, while the predictive power of Eq. (2) (CHI_{ACN1}) is lower, therefore the latest equation was not included in the QSAR studies. The CHI_{ACN2} and CHI_{MeoH} data correlated well with one another (n=18, r=0.979). Furthermore, the log P and C log P of cucurbitacins were calculated using ALOGPS program (Table 4).

TABLE 2
Linear equations and statistical data for the standard compounds and cucurbitacins^{a}

| | | | | | |
|---|---|---|---|---|---|
| Number | Compounds^{b} | Equation | r | S/O | RV |

CHI vs. t_{R}

| | | | | | |
|---|---|---|---|---|---|
| 2 | n = 7 (19-25) | CHI_{ACN1} = φ_{0ACN} = 3.9S3t_{R} − 5.473 | 0.962 | 0.13 | 25.79 |
| 3 | n = 9 (19-27) | CHI_{ACN2} = 6.172t_{R} − 42.993 | 0.998 | 0.03 | 2.68 |
| 4 | n = 10 (19-21, 23-29) | CHI_{MeOH} = φ_{0MeOH} − 6.951t_{R} + 2.046 | 0.996 | 0.03 | 1.84 |

C log P vs. CHI and biological data

| | | | | | |
|---|---|---|---|---|---|
| 5 | n = 18 (1-18) | CHI_{ACN2} = 21.495 C log P + 6.290 | 0.927 | 0.11 | 44.11 |
| 6 | n = 18 (1-18) | CHI_{MeOH} = 7.252 C log P + 45.304 | 0.959 | 0.08 | 2.69 |
| 7 | n = 17 (1-9, 11-18) | log IC₅₀ = −0.553 C log P + 3.052 | 0.742 | 0.21 | 0.15 |

log P vs. CHI and biological data

| | | | | | |
|---|---|---|---|---|---|
| 9 | n = 18 (1-18) | CHI_{ACN2} = 20.769 log P − 3.985 | 0.920 | 0.11 | 47.99 |
| 10 | n = 18 (1-18) | CHI_{MeOH} = 6.896 log P + 42.261 | 0.937 | 0.10 | 4.08 |
| 11 | n = 17 (1-9, 11-18) | log IC₅₀ = −0.688 log P + 3.905 | 0.948 | 0.10 | 0.03 |

log IC₅₀ vs. CHI

| | | | | | |
|---|---|---|---|---|---|
| 12 | n = 17 (1-9, 11-18) | log IC₅₀ = −0.026CHI_{ACN2} + 3.264 | 0.824 | 0.17 | 0.11 |
| 13 | n = 17 (1-9, 11-18) | log IC₅₀ = −0.083CHI_{MeOH} + 6.996 | 0.847 | 0.16 | 0.10 |

^{a}All equations show α < 0.01 for the F- and t-test.
^{b}Compounds identification number is indicated in parentheses.

TABLE 3
Standard mixtures chromatographic hydrophobicity indexes in buffered
ace-tonitrile and methanol using three different approaches^{a}

| Standard compound | CHI_{ACN}^{b} | CHI_{ACN}^{c} | CHI_{MeOH}^{b} |
|---|---|---|---|
| Theophylline (19) | 32.63 ± 0.07 | 15.76 | 25.76 ± 0.05 |
| Aniline (28) | — | — | 29.94 ± 0.05 |
| Benzimidazole (20) | 43.18 ± 0.12 | 30.71 | 41.07 ± 0.04 |
| Acetophenone (21) | 61.93 ± 0.05 | 64.90 | 52.47 ± 0.05 |
| Colchicine (26) | — | 41.37 | 57.56 + 0.04 |
| Indole (22) | 67.73 + 0.13 | 69.15 | — |
| Propiophenone (23) | 71.72 + 0.15 | 78.41 | 60.41 + 0.04 |
| Ph-theophylline (27) | — | 52.04 | 61.61 ± 0.04 |
| Butyrophenone (24) | 79.32 ± 0.14 | 88.49 | 66.79 ± 0.05 |
| Bromobenzene (29) | — | — | 69.43 + 0.07 |
| Valerophenone (25) | 86.66 ± 0.08 | 97.67 | 73.05 + 0.12 |

^{a}All data has less than ±1% error.
^{b}Isochratic and gradient elution of standard mixture.
^{c}Gradient elution of standard mixture and correlation with published data.

TABLE 4
Cucurbitacins cytotoxicity on HepG2 cells, chromatographic hydrophobicity
indexes in buffered acelonitrile and methanol using three different
approaches, and the software estimated C log P values^{a}

| Compound | IC₅₀ (μM) | CHI_{ACN1} | CHI_{ACN2} | CHI_{MeOH} | C log P | Log P |
|---|---|---|---|---|---|---|
| I-Gluc | 390.0 ± 10.0 | 46.48 | 37.50 | 56.41 | 1.84 | 2.09 |
| E-Gluc | 226.7 ± 15.3 | 53.98 | 49.13 | 62.27 | 2.75 | 2.28 |
| D | 77.3 + 8.7 | 58.31 | 55.83 | 60.40 | 2.05 | 312 |
| iso-D | 80.3 + 3.5 | 60.59 | 59.37 | 62.27 | 2.22 | 3.07 |
| I | 15.8 + 6.7 | 63.27 | 63.53 | 63.86 | 2.44 | 3.33 |
| I-Me | 15.0 + 5.6 | 64.84 | 65.95 | 66.58 | 2.69 | 3.81 |
| L-Me | 19.0 ± 1.0 | 64.84 | 65.95 | 66.58 | 3.55 | 3.79 |
| I-Et | 5.5 ± 0.5 | 69.99 | 73.94 | 69.61 | 3.08 | 4.15 |
| B | 27.7 ± 9.0 | 70.93 | 75.40 | 67.33 | 2.96 | 3.69 |
| iso-B | — | 72.86 | 78.38 | 68.50 | 3.12 | 3.68 |
| I-iPr | 7.0 ± 1.0 | 73.87 | 79.94 | 71.55 | 3.38 | 4.54 |
| I-aPr | 5.0 + 0.5 | 75.92 | 83.13 | 72.88 | 3.6 | 4.52 |
| F | 15.3 + 4.2 | 75.92 | 83.13 | 69.6! | 3.35 | 3.72 |
| E-Me | 12.0 ± 3.0 | 77.84 | 86.09 | 71.55 | 3.59 | 4.15 |
| E-Et | 5.1 + 0.9 | 82.76 | 93.73 | 74.25 | 3.98 | 4.68 |
| E-iPr | 4.3 + 0.5 | 86.84 | 100.04 | 76.30 | 4.29 | 4.78 |
| F-Me-Ac | 26.0 ± 1.0 | 86.84 | 100.04 | 76.30 | 4.30 | 4.29 |
| E-nPr | 3.7 ± 0.1 | 88.34 | 102.37 | 77.66 | 4.51 | 4.93 |

^{a}All CHI values has less than ±1% error.

It has been reported that CHI values depend on the type of stationary phase, the type of organic phase and, for acidic or basic compounds, the pH. The pH affected only the elution of benzimidazole, one of the compounds from the standard mixture; therefore, we employed buffered mobile phase to measure correctly the hydrophobicity. We recommend the selected test mixture, compounds 19-21, and 23-29, for the calibration of any 150 mm long RP-HPLC C18 column to measure CHI_{MeOH}. This standard mixture covers a range of CHI between 25 and 73. However, shorter columns are more convenient for less polar or larger compounds. For the CHI_{ACN} measurement of the standard mixture, Valkó et al. (Anal. Chem. 69) applied ODS-2 Interstil column of 150 mm. We chose Alltima C18 column of 250 mm and so we generated different values for these compounds (Table 3). This indicates that the column parameters have influence over the data. Nevertheless, any column can be calibrated by applying known CHI values for the standard compounds at fast gradient elution. Thus, CHI_{ACN1} translates the standard mixture and cucurbitacins lipophilicity on our column, while CHI_{ACN2} gives the calibrated values against published data for inter-laboratory purposes.

The cytotoxicity of 17 cucurbitacin analogues on HepG2 cells is listed in Table 4. This is believed to be the first in vitro assay of cucurbitacins on HepG2 cells to study the effect of structure alteration on cellular toxicity. Cells were challenged with cucurbitacins at various concentrations for a day and then live cells quantified with MTT dye. This period of time measures exclusively compounds cytotoxicity, while longer incubation time may lead to interference from metabolites. We did not have enough amount from iso-cucurbitacin B (compound 10) to include it into the biological assay.

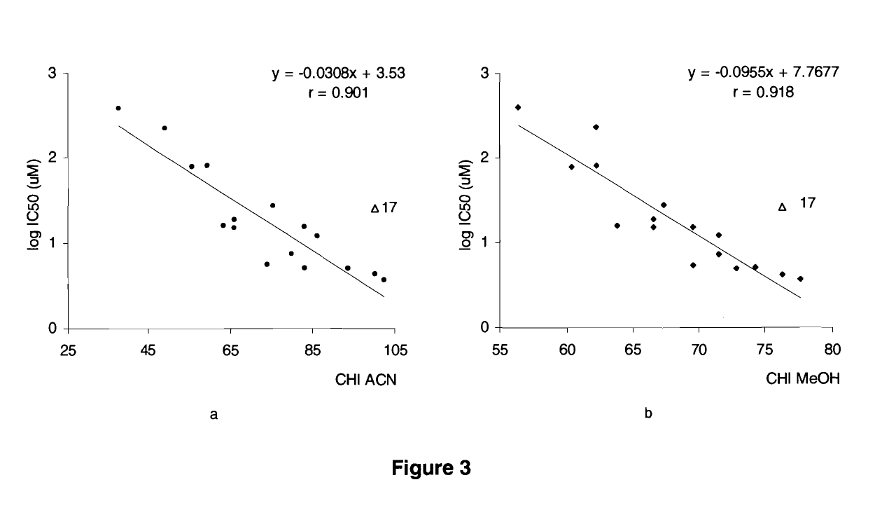

Correlations between CHI_{ACN2} or CHI_{MeOH} and logarithmic IC₅₀, as a measure of cytotoxicity, have been investigated (FIG. 3), and found statistically significant correlations (Table 2). These equations suggest that compounds lipophilicity increases in vitro cytotoxicity, with the exception of cucurbitacin E-Me-Ac (compound 17). This compound lipophilicity is increasing while its toxicity is decreasing relative to cucurbitacin E and E-Me ether analogues. Acetylation of C-16 hydroxyl diminishes toxicity in accordance with published data. Equations on FIG. 3 present the improved QSAR when 17 was not considered. Compounds 1 and 2 showed much lower toxicity (Table 4) than their aglycon counterparts, cucurbitacins I and E (compounds 5 and 13). It should be associated with the glucose molecule, which increases greatly both the polarity and the volume of the structure. Contrary to the in vivo data mentioned above, we noticed an increase in cytotoxicity for the alkylated derivatives on HepG2 cells. Additionally, cytotoxicity increased proportionally with increasing alkyl chain at C2 hydroxyl (compounds 6, 8, 11. 12, 14-16, and 18).

Good correlations were found between log P or C log P and CHI, and between log P or C log P and log IC₅₀ (Table 2). While the RP-HPLC hydrophobicity data is experimental, it confirms the good quality of the estimated octanol/water partition data. Overall, C log P shows better correlation with both CHI_{ACN2} and CHI_{MeOH}, and the log P correlates better with log IC₅₀. As mentioned above, different mathematical approaches were used to calculate log P or C log P. In addition, the log P values were reported to be more accurate than C log P. While log P correlates better than CHI with log IC₅₀, estimated lipophilicity is usually not as reliable as measured values. More research is necessary to validate the log P values calculated with the ALOGPS program. The scale of hydrophobicity defined as CHI_{ACN}, CHI_{MeOH}, log P or C log P (Table 4) indicates that CHI_{ACN} has the largest range, and therefore it should provide a highly sensitive measure, allowing more discrimination among similar compounds. Yet CHI_{ACN} is not correlating the best with the cytotoxicity. The steroid-like cucurbitacins diffuse through the biological membrane by nonmediated transport. Only the presence of C19 methyl group at position 9 instead of the usual position 10 for steroids differentiates the cucurbitacin skeleton from steroids. Consequently, the more lipophilic compounds can cross the lipid bilayer easier than their polar homologues, leading to differentiation in their partitioning between the media and cells. Lipophilicity also plays a dominant role in ligand-receptor interactions, e.g. in binding drug to the target molecule inside the cell. We may speculate that cytotoxicity of cucurbitacins involves hydrophobic interaction with the target molecule within the cell, and analogues with higher lipophilicity may have stronger interaction. Furthermore, it has been reported that cucurbitacins are activated within several hours in the cytoplasm and only their metabolites are implicated in the mechanism of action If the metabolites are involved in the interaction, their hydrophobicity may proportionally change with the hydrophobicity of the original compound, demonstrated by the strong relationship between lipophilicity and cytotoxicity.

Conclusion

RP-HPLC is a fast, high-throughput and highly precise technique to determine compound hydrophobicity, which is an important descriptor in drug design. Cucurbitacins CHI indicates a wide range of lipophilicity. The ACN mobile phase leads to a better resolution and wider range of CHI data than MeOH. On the other hand, a shorter HPLC column generates more accurate data than a longer column. High correlations have been found between the software-estimated log P or C log P and CHI, which validates the estimated lipophilicity data. Overall, lipophilicity increases the basal toxicity of cucurbitacins on HepG2 cells. The presence of Δ^{1,2} generally increases toxicity. The extension of R₁ alkyloxy chain or acetylation of C25- OH increases lipophilicity as well as toxicity. The alkylation of diosphenol increases toxicity on HepG2 cells, in opposite to the lower toxicity demonstrated by others in animals. While the trend is true for most analogues, acetylation of C16-OH group leads to relatively higher lipophilicity but lower toxicity.

In summary, drug development of cucurbitacins is focused on derivatives that have lower cytotoxicity. Therefore, the effect of structural modification on in vitro cytotoxicity has been investigated. Lipophilicity or chromatographic hydrophobicity index (CHI) was chosen as the molecular property. CHI was determined by RP-HPLC in both aqueous acetonitrile and aqueous methanol. Compounds CHI range was wide and better defined in acetonitrile (CHI_{ACN}=46-88 and 38-102) than in methanol (CHI_{MeOH}=56-78). Higher resolution was achieved in acetonitrile, and higher precision on the shorter C18 column. Cucurbitacins cytotoxicity (IC₅₀) was measured on the hepatocyte-derived HepG2 cells. Strong relationship between CHI and logarithmic IC₅₀ was found. As a result, cytotoxicity increased linearly with increasing hydrophobicity (r≧0.90). Other lipophilicity parameters, such as log P and C log P were also estimated. Cytotoxicity correlated well with log P (r=0.95) and slightly with C log P (r=0.74). The log P and C log P data showed good correlation with CHI (r>0.92). Overall, alkylation of C1 hydroxyl, unsaturation of C₁-C₂ bond, and acetylation of C25 hydroxyl increased both lipophilicity and cytotoxicity. This assay should prove useful for monitoring cucurbitacin homologues or other drug candidates for their cytotoxicity.

### Example 2

Interferon-based therapy is a standard treatment in modern medicine for chronic viral hepatitis and its use is associated with the risk of relapse and danger of side effects. Ribavirin, corticosteroids, nucleoside analogues and thymosin are the usual additives to this treatment. Various categories of compounds isolated from natural sources have been evaluated for the treatment of hepatocellular injury.

The human hepatoma HepG2 cell line provides an appropriate in vitro model for the assessment of likely hepatotoxicity in vivo. It has the biosynthetic capability of normal liver parenchymal cells often lost by primary hepatocytes, and it secretes the major plasma proteins. In addition, HepG2 is one of the 3 cell lines to be used in the chemical and pharmaceutical industries to evaluate toxicity of new chemicals on humans.

It has been well documented that hepatic stellate cells play a central role in liver fibrogenesis in experimental models of liver fibrosis as well as in human chronic liver disease. Its activation is characterized by the elevated proliferation rate, loss of vitamin A storage, expression of α-smooth muscle actin, and synthesis and excretion of some extracellular matrix components.

Cucurbitacins from cucurbit species are a class of triterpenes and have been used in traditional medicine for a long time for liver treatments. Cucurbitacins were known for their potent and differential cytotoxicity and listed on the top of the most cytotoxic compounds at NIH—NCI cancer research program. Although cucurbitacins showed potent cytotoxicity, selective anticancer activity for prostate, brain and ovarian cancers, they are less toxic to liver cell.

Hepatoprotective (liver protection) and anti-proliferative activities of cucurbitacins were investigated using HepG2 and HSC-T6 cell lines. Silybin (known liver protective drug) was used for comparison.

We are documenting here for the first time the following:

1. Hepatoprotective effect of cucurbitacin compounds. Our finding documented for the first time that cucurbitacins analogues isolated and prepared in our lab protect liver against hepatotoxicity at dose of 0.5 and 0.2 times the toxic dose and induced marked increase in cell viability. Cucurbitacins E and I glucosides show significant protection over the cells from which the first one has similar EC₅₀ value to silybin. Some aglycons show significant protection even at the level of 0.2 IC₅₀, while alkyl groups bigger than the methyl at C2 position decreases the activity or turns compounds to toxic ones. The presence or absence of Δ^{1,2}, Δ^{23,24} or that of the C25 acetyl group doesn't affect significantly the activity.

2. Anti-proliferative Effect of cucurbitacin compounds. Our finding documented for the first time that cucurbitacins analogues isolated and prepared in our lab showed a potent anti-proliferative effect against hepatic stellate cells (HSC-T6). Cucurbitacin I gluc, E gluc, D, iso-D, I, I-Me, L-Me, B, and E were proved to be good candidates for further drug development. Glucosides indicate no toxicity (IC₅₀>50 μM) on the cell line.

Conclusion

We are documenting that cucurbitacin analogues isolated and prepared in our lab showed a significant protective activity against the hepatotoxic effect of CCl₄ on HepG2 cells even at 0.2 IC₅₀ level (Table 5). The same compounds also demonstrate potent antiproliferative effect against hepatic stellate cells.

TABLE 5
Cucurbitacins (Structure modification achieved at
R₁, R₂, R₃, R₄, C1, C2, C3 and C23-C24)

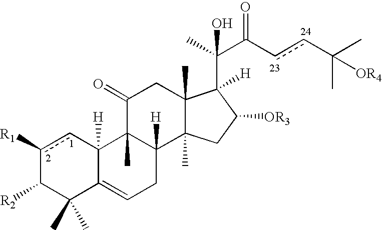

| Cucurbitacin | R₁ | R₂ | R₃ | R₄ | Other | IC₅₀ (μM) |
|---|---|---|---|---|---|---|
| I Gluc^{a} | Glu | ═O | H | H | Δ^{1,2}, Δ^{23,24} | 390.0 ± 10.0 |
| E Gluc^{a} | Glu | ═O | H | Ac | Δ^{1,2}, Δ^{23,24} | 226.7 ± 15.3 |
| D | OH | ═O | H | H | Δ^{23,24} | 77.3 ± 8.7 |
| Iso-D | ═O | OH | H | H | Δ^{23,24} | 80.3 ± 3.5 |
| I | OH | ═O | H | H | Δ^{1,2}, Δ^{23,24} | 15.8 ± 6.7 |
| I-Me | O-Me | ═O | H | H | Δ^{1,2}, Δ^{23,24} | 15.0 ± 5.6 |
| L-Me | O-Me | ═O | H | H | Δ^{1,2} | 19.0 ± 1.0 |
| I-Et | O-Et | ═O | H | H | Δ^{1,2}, Δ^{23,24} | 5.5 ± 0.5 |
| B | OH | ═O | H | Ac | Δ^{23,24} | 27.7 ± 9.0 |
| I-iPr | O-iPr | ═O | H | H | Δ^{1,2}, Δ^{23,24} | 7.3 ± 0.6 |
| I-nPr | O-nPr | ═O | H | H | Δ^{1,2}, Δ^{23,24} | 5.0 ± 0.5 |
| E | OH | ═O | H | Ac | Δ^{1,2}, Δ^{23,24} | 15.3 ± 6.6 |
| E-Me | O-Me | ═O | H | Ac | Δ^{1,2}, Δ^{23,24} | 12.0 ± 3.0 |
| E-Et | O-Et | ═O | H | Ac | Δ^{1,2}, Δ^{23,24} | 5.1 ± 0.9 |
| E-iPr | O-iPr | ═O | H | Ac | Δ^{1,2}, Δ^{23,24} | 4.3 ± 0.5 |
| E-Me-Ac | O-Me | ═O | Ac | Ac | Δ^{1,2}, Δ^{23,24} | 26.0 ± 1.0 |
| E-nPr | O-nPr | ═O | H | Ac | Δ^{1,2}, Δ^{23,24} | 3.7 ± 0.1 |

^{a}β-D-glucopyranose

### Example 3

Cucurbitacin Hepatoprotective Activity

The heptoprotective active of cucurbitacin analogs (listed in Table 1 and Table 5) in vitro is explored in this example. Two liver cell lines were selected, the human hepatocyte-derived HepG2 cells and the rat liver stellate cells-derived HSC-T6 cells. HepG2 cells are a useful in vitro model for investigation of the toxicity of drugs, since HepG2 cells retain many of the specialized functions characteristic of normal human hepatocytes. Cucurbitacin cytoprotection against CCl₄ toxicity was specifically examined on these cells.

Stellate cells play important role in liver fibrosis. Upon liver injury, stellate cells become activated and start to proliferate without control leading to fibrosis and later cirrhosis. Cucurbitacin anti-proliferative assay was conducted on activated HSC-T6 cells.

Platelet-derived growth factors (PDGF) and fetal bovine serum (FBS) were studied to determine their activating effect of HSC-T6 proliferation. Experimental conditions were optimized on both cell lines using silybin, the well known hepatoprotective and antifibrotic compound. To have more insight into the mechanism of liver protection, this work also involves the study of cucurbitacin antioxidant and anti-inflammatory activities.

Cucurbitacin Cytoprotection Activity on HepG2 Cell Line

Optimization of HepG2 Cell Growth

HepG2 cells have the tendency to pile up, shrink and cluster rather than spread nicely across the plate or flask. Accordingly, cells form smaller and larger clusters and their proliferation slows down tremendously. The ATCC scientific group suggested using their special media formulated for HepG2 cells to attempt a better spread of the cells. There is no other growth media available that ensures continuous optimal cell growth conditions.

Our experiments on HepgG₂ cells faced challenges when cells aggregated for longer periods of time (days or weeks) and did not spread evenly during cell growth. We managed to optimize cell growth conditions by changing the cell growth media ingredients. These modifications included change of serum: lot, company, or type (FBS vs. newborn calf serum or chicken serum), heat-inactivated serum vs. not heat-inactivated serum, lack or presence of antibacterial or antimycotic material, or change of media type (EMEM vs DMEM vs RPMI). Our cytoprotective screening experiments were conducted on healthy HepG2 cells.

Effect of Hepatotoxins on the Viability of HepG2 Cells

The activity of two toxins was studied first on HepG2 cells—bromobenzene (BB) and CCl₄. Cells were challenged with the toxins at different concentration levels. Precipitate formation was observed when BB was diluted with media prior to addition to the cells; therefore, both the precipitate and the supernatant were added separately over cells to study their toxic effect. At concentration of 20 nM BB, cells showed no viability while at lower concentrations the toxic effect was inconsistent. On the other hand, CCl₄ cytotoxicity grew in dose-dependent manner. At 4.5×10⁻³ M, CCl₄ reduced the cell viability to 40%-50%. Because of this, CCl₄ was chosen to study the protective activity of cucurbitacin analogs.

Cytoprotective Effect of Silybin

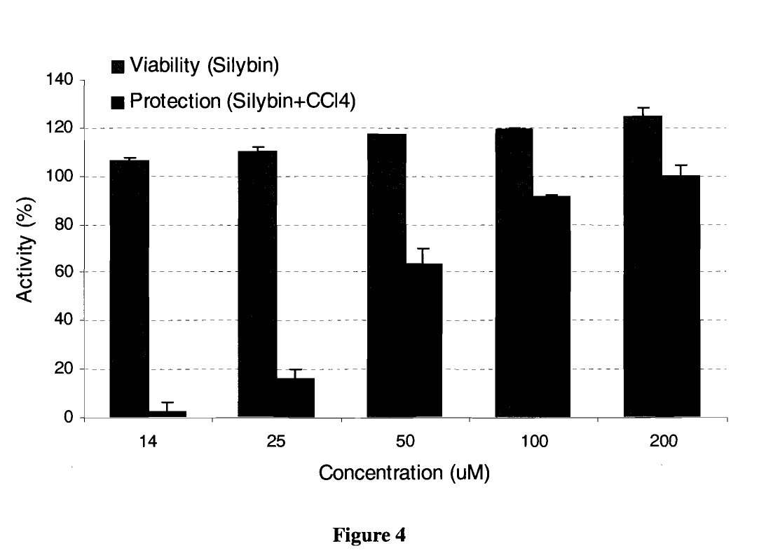

Experimental conditions were standardized on silybin. Cells were challenged with CCl₄ (4.5×10⁻³M) in presence of silybin at various concentrations. The effect of silybin alone was also monitored on cells (FIG. 4) and it indicated no toxicity at these concentrations (14 to 200 μM)—it actually improved cell growth up to 125%. Silybin shows high cytoprotection and completely protects cells from the toxic effect of CCl₄ at concentration higher than 100 μM. The EC₅₀ (the molar concentration of the compound, which produces 50% of the maximum possible cell protection against CCl₄ effect) was calculated from the logarithmic correlation between concentration and activity and found to be 45 μM.

Cytoprotective Effect of Cucurbitacin

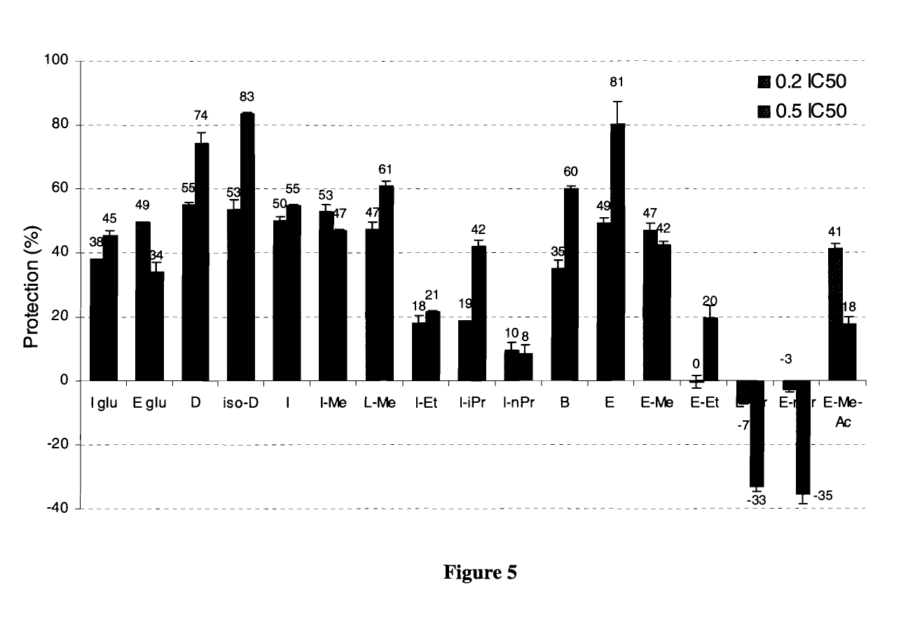

Cucurbitacin analogs (please see Table 1 and Table 5) cytoprotection against CCl₄ toxicity was determined at two concentration levels—at 20% and 50% of their IC₅₀ values (Table 5). In most cases, cytoprotection was found to be higher at 50% IC₅₀ concentration level. The majority of compounds indicated good protection (≧50%) on cells as cucurbitacin E glucoside (E-Gluc), D, iso-D, I, I-Me, L-Me, B and E (FIG. 5). Particularly, cucurbitacin D, iso-D and E yielded high cytoprotection (74%-83%) against CCl₄ toxicity. The EC₅₀ value was estimated for these compounds and together with their IC₅₀ values they are listed in Table 6. The ratio of IC₅₀-to-EC₅₀ (hereafter T/A) is also indicated in this table, which gives the margin between toxic and cytoprotective concentrations. A higher T/A value represents a higher safety margin for the compound. Other derivatives show less cytoprotection (<50%) as cucurbitacin I glucoside (I-Gluc), I-Et, I-iPr, I-nPr, E-Me, E-Et, and E-Me-Ac (FIG. 5). On the other hand, cucurbitacin E-iPr and E-nPr increase the toxicity of CCl₄ on the cells.

Several cucurbitacin cytoprotective activity was measured at four concentration levels (50%, 20%, 12.5% and 6.25% of their IC₅₀ values), which demonstrated increased activity while increasing concentration (Table 6). Exception is made by cucurbitacin E glucoside, which shows higher cytoprotection at ⅕ IC₅₀ than at ½ IC₅₀. Furthermore, the activity of cucurbitacin I does not show linear trend and it levels off at around ⅕ IC₅₀. For the rest of the compounds from Table 6 the dose—response relationship is linear (r>0.94).

TABLE 6
Cucurbitacin derivatives cytoprotection on HepG2 cells at four concentrations

| IC₅₀ | E gluc | Cuc I | Cuc I-iPr | Cuc B | Cuc E | Cuc E-iPr |
|---|---|---|---|---|---|---|
| 50% | 33.92 + 2.92 | 54.75 ± 0.29 | 41.73 ± 2.06 | 60.01 ± 0.80 | 80.55 ± 6.80 | −32.99 ± 1.70 |
| 20% | 49.44 ± 0.29 | 50.09 ± 1.02 | 18.69 ± 0.13 | 34.92 ± 2.76 | 49.21 ± 1.73 | −7.00 ± 0.17 |
| 12.5% | 17.96 ± 1.14 | 17.50 ± 1.32 | 4.15 ± 0.48 | 25.25 ± 1.36 | 21.72 ± 0.75 | −7.81 ± 0.68 |
| 6.25% | −2.36 ± 1.08 | 4.55 ± 0.64 | −17.10 ± 1.54 | 12.45 ± 1.11 | 12.41 ± 0.78 | −0.60 ± 1.11 |

TABLE 7
Cucurbitacin cytoprotection (EC₅₀) on
HepG2 cells against CCl₄ toxicity

| | EC₅₀ | IC₅₀ | | | EC₅₀ | IC₅₀ | |
|---|---|---|---|---|---|---|---|
| Compound | (μM) | (μM) | T/A^{a} | Compound | (μM) | (μM) | T/A |
| E Glu | 45.3 | 226.7 | 5.0 | L-Me | 5.0 | 19.0 | 3.8 |
| D | 9.0 | 77.3 | 8.6 | B | 10.5 | 27.7 | 2.6 |
| i-D | 13.8 | 80.3 | 5.8 | E | 3.2 | 15.3 | 4.8 |
| I | 3.2 | 15.8 | 5.0 | E-Me | 2.4 | 12.0 | 5.0 |
| I-Me | 5.3 | 15.0 | 2.9 | E-Me-Ac | 5.2 | 26.0 | 5.0 |

^{a}T/A is the ratio of IC₅₀ and EC₅₀

Cucurbitacin Anti-Proliferative Activity on HSC-T6 Cell Line

PDGF-Driven Proliferation

The proliferation assay described by Zhang et al. (Zhang et al., Acta Pharmacol. Sin. 21: 253-256, 2000), was followed to determine the proliferative effect of PDGF on HSC-T6 cells, and yielded to total cell confluency prior to the addition of PDGF to the cells. Therefore, experimental conditions were changed stepwise to reduce cell confluency. When cell concentration was reduced from the initial 10,000 to 4,000 cells/well, and FBS concentration was reduced from 10 to 2%, cell confluency reached approximately 50% prior to the addition of PDGF. However, PDGF did not have any proliferative effect on the cells. Further modifications to the initial assay were done such as increase of period of incubation or increase of PDGF concentration. Cells incubation with PDGF for 2 days indicated some degree of proliferation (0-22%), but it was not reproducible from one trial to the other. In addition, PDGF concentrations higher than 10 ng/ml such as 20 and 50 ng/ml did not induce proliferation relative to control.

The experimental conditions presented by Yang et al. (Yang et al., World J. Gastroenterol. 9: 2050-2053, 2003) were also examined. It involved 1-day cell starvation in serum-free media followed by 3 h challenge with drug candidates and then 2 days incubation with PDGF in serum-free media. In our study, PDGF was added at various concentration levels to starved cells and incubation period with PDGF was increased to 2-to-3 days. However, we could not achieve a stable and significant level of cell proliferation in the presence of PDGF. Additionally, we employed human as well as rat PDGF in our studies, which did not improve the proliferation. Proliferation of HSC-T6 cells in all these trials was not evident in presence of PDGF.

Serum-Driven Proliferation

The proliferation effect of serum on HSC-T6 cells was optimized and cucurbitacin antiproliferative activity was measured. Serum contains a large number of growth factors, hormones and other nutrients, which help cells to grow and multiply. Several trials were conducted. In experiment 1, cells were challenged with cucurbitacin analogs for 4 h and then cells were grown in drug-free media for two additional days.

The compounds decreased cell proliferation in a dose dependent manner. The EC₅₀ was calculated for the majority of compounds (Table 8). It could not be estimated for silybin, cucurbitacin E glucoside, I glucoside, and I-nPr, because their antiproliferation activity did not reach 50%.

To overcome this problem, the amount of incubation time was increased to 24 h, and cucurbitacin activity measured on starved (Exp. 2) or proliferative (Exp. 3) cells (Table 8). Cells turned quiescent when serum was not supplemented for one day (Exp. 2). Activity of a few compounds was monitored on quiescent cells, and their activity compared to the ones measured on proliferating cells (Exp. 3). As a result, cucurbitacin and silybin activity was found to be lower on quiescent cells (higher EC₅₀ values) than on proliferating cells.

TABLE 8
Cucurbitacin cytotoxicity and inhibition activity on HSC-T6

| | Experi- | Experi- | Experiment 3 | | |
|---|---|---|---|---|---|
| | ment 1 | ment 2 | IC₅₀ | EC₅₀ | |
| Compound | EC₅₀ (μM) | EC₅₀ (μM) | (μM, T) | μM, A) | T/A ^{c} |
| Silybin | ˜20% ^{a} | 68 | non | 18.83 | — |
| | | | toxic | | |
| I gluc | ˜20% ^{a} | — | 256.0 | 4.15 | 64 |
| E gluc | ˜46% ^{a} | 20.0 | 102.0 | 3.28 | 29 |
| D | 3.80 | — | 26.0 | 0.07 | 344 |
| iso-D | 2.13 | — | 8.7 | 0.06 | 139 |
| I | 0.36 | — | 3.6 | 0.02 | 150 |
| I-Me | 3.40 | 1.4 | 6.4 | 0.11 | 62 |
| L-Me | 10.80 | 1.0 | 6.7 | 0.18 | 31 |
| I-Et | 7.95 | 1.4 | 3.5 | 0.34 | 10 |
| I-iPr | 3.85 | — | 2.5 | 0.25 | 9 |
| I-nPr | ˜18% ^{b} | — | 2.5 | 0.33 | 8 |
| B | 1.18 | — | 4.4 | 0.02 | 180 |
| E | 0.86 | — | 2.0 | 0.04 | 54 |
| E-Me | 3.05 | — | 2.8 | 0.08 | 33 |
| E-Et | 5.60 | — | 3.5 | 0.27 | 13 |
| E-iPr | 2.13 | — | 2.5 | 0.32 | 8 |
| E-nPr | 2.28 | — | 2.8 | 0.34 | 8 |
| E-Me-Ac | 24.20 | — | 11.5 | 0.91 | 12 |

^{a} Compounds anti-proliferation activity measured at 100 μM;
^{b} Cucurbitacin I-nPr anti-proliferation activity measured at 2-6 μM.
^{c} T/A is toxic over antiproliferation concentration, defined in IC₅₀ and EC₅₀, respectively;

Cytotoxicity of all cucurbitacin analogs was additionally measured in experiment 3 (Table 8). Data indicates significant differences between toxic and active concentration levels. Very high T/A values (>100) are found for cucurbitacin D, iso-D, I, and B. Medium to high T/A values are found for cucurbitacin I glucoside, E glucoside, I-Me, L-Me, E, and E-Me. Lower T/A values are recorded for the Et-, iPr-, -nPr derivatives of cucurbitacin I and E, and for E-Me-Ac.

HepG2 and HSC-T6 Cells Morphology

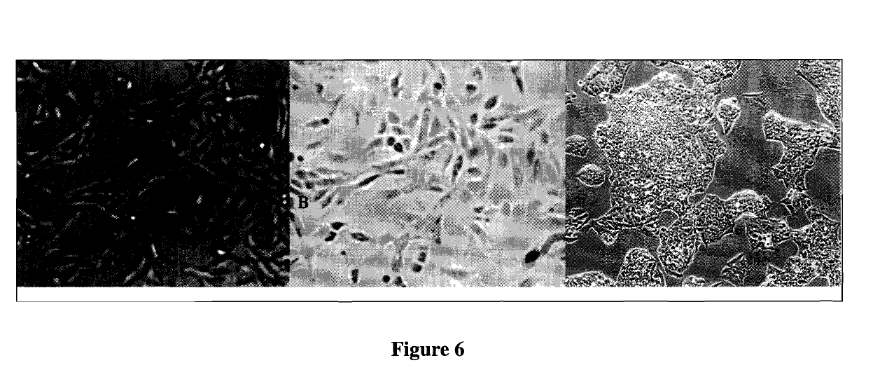

Healthy HSC-T6 stellate cells exhibit an activated phenotype as reflected in their fibroblast-like (spindle) shape and rapid proliferation in monolayer culture. Normal HepG2 cells are less angular and do not have clearly defined subcellular structures. These adherent cells grow in three-dimensional clusters. The empty space between cellular clusters is normal even for a highly dense population. Both cell types are presented in FIG. 6.

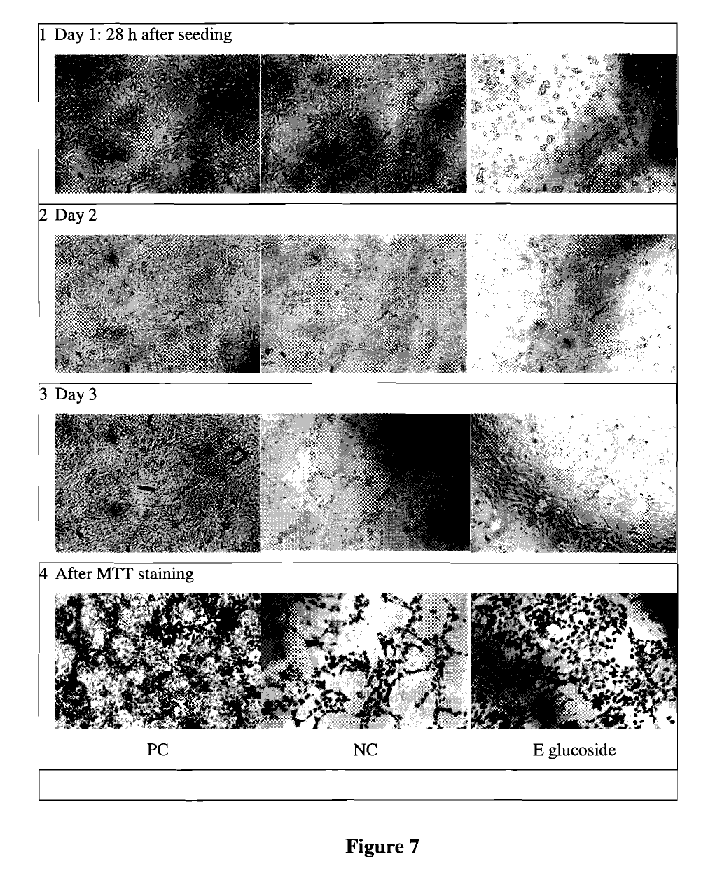

HSC-T6 cells were photographed under phase-contrast microscope to record possible changes in cell morphology during experiment 1. Cells challenged with cucurbitacin E glucoside, positive control (PC), and zero control (ZC) are illustrated in FIG. 7. We are monitoring specifically the effect of compound on the cells, cell recovery after 4 h challenge, and relative cell proliferation. E glucoside led to some degree of cellular alterations: HSC-T6 cells will round up, shrink, and lose intercellular adhesion, without detaching from the well (row 1, FIG. 7). On the other hand, the standard compound silybin does not induce morphological changes during its incubation with the cells. After a 4 hour challenge with each cucurbitacin, media was refreshed and within 24 hours cells showed recovery from the alteration induced by the glucoside (row 2, FIG. 7). Within 24 hours cells turn quiescent in the ZC well, and their viability relative to the PC was found to be about 30% on day 2 and 60% on day 3 of incubation. Live cells are quantified at the end of the assay (row 4) with MTT dye. The largest amount of cells is observed in the PC, lesser amount in the sample well, and the least amount in the ZC well. In this particular case E glucoside shows about 38% anti-proliferation activity.

After challenging with CCl₄, HepG2 cells showed morphological alterations. Although cells were still attached to the bottom of the well, they rounded up, shrank, and separated from each other. When cucurbitacin were added to the cells together with CCl₄, cell viability significantly improved while morphologically cells remained in altered state. Cucurbitacin alone at ½ IC₅₀ or ⅕ IC₅₀ altered cell morphology, cells rounded up and shrank similarly to HSC-T6 cells when these were challenged with cucurbitacin in experiment 1 (FIG. 7). Significant improvement in cell shape and size was noticed when toxin was added to cells in the presence of silybin. Complete cytoprotection was achieved when silybin concentration was higher than 100 μM, and cells looked healthy and similar to the positive control cells.

Antioxidant and Anti-Inflammatory Activities

Cucurbitacin antioxidant activity was studied first by the DPPH® Stable Free Radical Scavenging Assay (Cotelle et al., Free radical Biology & Medicine 20(1): 35-43, 1996). Experimental conditions were optimized on the standard compound ascorbic acid. Data indicated 30% activity at 50 μM concentration, and 100% activity at 100 μM for ascorbic acid. While the original assay required 10 minutes incubation time for the reaction to occur, this time was increased up to 30 minutes in case cucurbitacin were reacting slower than other reagents. Cucurbitacin B at 50 μM did not indicate any activity.

Cucurbitacin antioxidant activity was also studied by the ABTS®⁺ Radical Cation Decolorization Assay (Re et al., Free radical Biology & Medicine 26(9/10): 1231-1237, 1999 and Pellegrini et al., Methods in Enzymology 299: 379-389, 1999). The assay was validated on the standard compound trolox, with 50% inhibition at 0.58 mM. Cucurbitacin B activity was monitored at 50 μM, 100 μM, and 1.8 mM. The time interval was expanded up to 2 hours in case cucurbitacin B was reacting slowly. No activity was observed.

Cucurbitacin anti-lipid peroxidation was studied by the Microsomal Lipid Peroxidation Assay (Engineer et al., Biochemical Pharmacology 38(8): 1279-1285, 1989). Lipid peroxidation of microsomal suspension was induced both enzymatically and chemically and validated on the standard compound quercetin. It inhibited lipid peroxidation in a dose dependent manner showing 100% inhibition at 0.1 mM quercetin in both chemical and enzymatic assays. Activity of 3 cucurbitacin analogs, B, E, and E-Me-ether was monitored at 0.1 mM indicating no inhibition in either the chemical or enzymatic assay.

Cucurbitacin anti-inflammatory activity was monitored by the Anti-Hyaluronidase Assay (Facino et al., Il Farmaco 48: 1447-1461, 1993 and Linker, A., Hyaluronidase. In: Methods of enzymatic analysis. Vol. 4., Bergmeyer, H. U. (Editor), Verlag Chemie GmbH, Berlin, 256-262, 1984). This assay was validated using the standard compound phenylbutazone. At 71.68 mM it inhibited the enzyme activity by 50%. Cucurbitacin B showed no activity up to this concentration level.

Discussion

Cucurbitacin B, iso-B and E demonstrated protective and preventive activity by significantly reducing serum enzymes level, steatosis, inflammation, and experimental cholestasis. Fibrosis and cirrhosis were noticeably reduced as well. To the best of our knowledge, the hepatoprotective effect of cucurbitacin has not previously been investigated on cultured cells. Therefore, we investigated the activity of 17 cucurbitacin analogs on two different liver cell lines, HepG2 and HSC-T6 cell line. Cell lines offer the unique possibility to elucidate interactions with vital cellular functions such as metabolism, intercellular communication, signal transduction, cell growth and cell death that were formerly difficult to address in vivo. Furthermore, in vitro data provides a relatively quick and inexpensive way of ranking chemicals according to their biological activity.

Primary cultures of hepatocytes are the key tools in studying pharmacological and toxicological aspects of liver injury. Immortalized HepG2 cell line proved to be very useful in screening of natural products or xenobiotics in order to study toxicity, carcinogenesis, metabolism and cytoprotection. This hepatoblastoma-derived cell line expresses many of the functions attributed to normal hepatocytes or often lost by primary hepatocytes, and they have the biosynthetic capabilities of normal liver parenchyma cells.

Evaluation of Cucurbitacin Cytoprotection on HepG2 Cells

The effect of the hepatotoxin bromobenzene was examined first on HepG2 cells, since it was successfully used earlier. In our trials, bromobenzene formed precipitate with media and yielded inconsistent data. Our experiment involved EMEM cell growth media instead of the suggested DMEM media. In addition, it was not clear in the described assay whether the media contained serum when bromobenzene was added to it. Based on these, our media probably contained some additional ingredients that yielded to the precipitation of bromobenzene.

The hepatotoxic effect of CCl₄ was examined and conditions successfully optimized to use it as the toxic agent. While CCl₄ is often used in in vivo assays, its effect was never reported on HepG2 cells. We found a dose-dependent cytotoxicity for CCl₄ with cell viability of˜50% at 4.5 10⁻³ M. CCl₄ is one of the most intensively studied hepatotoxin in vivo. It causes centrolobular necrosis and associated fatty liver. In addition, it is nephrotoxic and a suspected carcinogen. There are several mechanisms studied by which CCl₄ exposure leads to liver injury. The major effects are lipid peroxidation, cytosolic Ca²⁺ increase, and activation of Kuppfer cells. Sustained elevation of intracellular Ca²⁺ has been associated with mithocondrial dysfunction, endonuclease activation, protease activation, phospholipase activation, and perturbation of cytoskeletal organization.

The majority of cucurbitacin analogs induced marked increase in cell viability against CCl₄ mediated cytotoxicity (FIG. 5). Highest activity was detected at half dose of IC₅₀. Particularly high protection (74-83%) was observed for cucurbitacin D, iso-D and E. Ten analogs EC₅₀ was successfully estimated (Table 7). The EC₅₀ value was found to be generally five times lower than the IC₅₀ dose, which implied some potential as cytoprotective agents. Although their activity was less than 50% and their EC₅₀ could not be estimated, cucurbitacin I glucoside, I-Et, 1-iPr, and E-Me-Ac indicated significant protection (>20%). Few compounds such as cucurbitacin I-nPr, E-Et, E-iPr, and E-nPr did not show protection or they increased the toxicity of CCl₄ on the cells (FIG. 5). The various cytoprotective activity levels are perhaps connected to the different structural characteristics of cucurbitacins.

Cucurbitacin cytoprotective activity was found to be within a narrow range. At higher concentration than ½ IC₅₀ they showed toxicity on cells and at lower concentration than ⅕ IC₅₀ their activity diminished significantly. The EC₅₀ of ten out of the 17 cucurbitacin analogs could be measured and found to be 5-fold less than the IC₅₀ concentration. While this margin is not large and cucurbitacin show toxicity on HepG2 cells, their toxicity on HeLa cells was found to be much larger. This differential cytotoxicity was demonstrated and it confirms that cucurbitacin are less toxic on HepG2 cells. The low margin between active and toxic dose was also indicated earlier on various cell lines or in vivo. Highest antitumor activity was found to be at ½ LD₅₀ (lethal dose) in vivo for a large number of cucurbitacin derivatives and at lower dose the activity diminished. Five-fold higher concentration from cucurbitacin D was required in vitro to produce similar changes in normal human lymphocytes to leukemic lymphocytes.

Cucurbitacin cytoprotective activity on HepG2 cells was found to be equal or better than the cytoprotective activity of the standard compound silybin (Table 6). While silybin does not show cytotoxicity on the cells and does not lead to morphological changes, cucurbitacin have considerable toxicity and yield morphological changes at EC₅₀ concentration. Cells' morphological alteration may be related to cucurbitacin effect on the cytoskeleton proteins. The alteration of cytoskeleton proteins was demonstrated earlier to be part of cucurbitacin mechanism of anticancer and antiinflammatory activity. Alteration of the cytoskeleton may disable cell growth. Furthermore, earlier findings indicated that the binding to glucocorticoid receptors did mediate cucurbitacin cell growth inhibitory effect on several cultured cell lines, including several hepatoma cell lines and HeLa cells. Alteration of the cytoskeleton or/and binding to the glucocorticoid receptor would explain why we did not reach 100% cytoprotection by cucurbitacin; on the other hand 100% protection was attained in presence of silybin.

Evaluation of Cucurbitacin Antiproliferation Effect on HSC-T6 Cells

It was well documented that hepatic stellate cells play a central role in liver fibrogenesis in experimental models of liver fibrosis as well as in human chronic liver disease. Stellate cells activation process is characterized by the elevated proliferation rate, loss of vitamin A storage, expression of α-smooth muscle actin, and synthesis and excretion of some extracellular matrix components. HSC-T6 is a well characterized immortalized hepatic stellate cell line. It expresses myogenic and neural crest cytoskeletal filaments. While it cannot replace primary cells for studying early dynamic events of cellular activation, it serves as a useful tool for studying hepatic stellate cell mechanism, biology, and drug candidates screening. In addition, primary stellate cells are labor-intensive to prepare and they can vary in quantity and phenotype. The HSC-T6 cells have stable phenotype, well characterized, and a large number of cells can be generated.

PDGF-Derived HSC-T6 Cell Proliferation

Although various experimental conditions were established regarding incubation time, concentration level and type of PDGF, we could not achieve a stable and significant level of cell proliferation in the presence of PDGF. The phenotypic transformation of HSC has been linked to some cytokines, including PDGF, and their intracellular signal transduction pathways have been largely characterized. The proliferative effect of PDGF is well documented on primary HSC cells, but not on the immortalized cell line HSC-T6. The induction of HSC proliferation occurs between 24 and 48 hours and reaches plateau at 48 hours. The onset of proliferation coincides with the induction of PDGF receptor expression. Pre-incubation of HSC with Kuppfer cell medium elicits the expression of PDGF receptors. When McFarland et al. studied the effect of PDGF on cloned turkey satellite cells and embryonic myoblasts, they obtained good results when adding PDGF to cells in the presence of basic fibroblast growth factor (bFGF). They also showed that the proliferation improved when PDGF was added together with bFGF and insulin-like growth factor- I (IGF-I), or insulin. This may suggest that PDGF alone does not induce proliferation, because it requires the presence of other growth factors. This explains the reason we did not achieve a considerable proliferation with PDGF.

Serum-Driven HSC-T6 Cell Proliferation

Cucurbitacin analogs demonstrated anti-proliferation effect on HSC-T6 cells proliferated in serum-supplemented media (Table 8). We established several experimental conditions such as 4 hours of incubation time with cucurbitacin, and 2 day cell incubation with fresh media after challenging with cucurbitacin (Exp. 1). Other experiments involved addition of cucurbitacin for 24 hours over starved cells (Exp. 2) vs. proliferating cells (Exp. 3), and one day incubation of cells with fresh media after challenge. The 4 hour incubation period with cucurbitacin triggered anti-proliferation effect. This effect, however, was hindered by a second cycle of cell proliferation during the two-day cell growth period (see silybin, E and I glucosides, and for I-nPr). Silybin did not show significant anti-proliferation activity when the cell growth period after challenge was reduced to one day. Because of that, the challenge time was increased to 24 hours (Exp. 2 and 3).

Cells turned quiescent during a day of incubation with serum-free media in experiment 2; this is demonstrated by the doubling of cell number in the PC wells relative to ZC. In experiment 3, only 40% of the cells divided. The ratio of EC₅₀ values for exp. 2 and exp. 3 varied between 3.6 and 12.7. This may indicate differential cytotoxicity of silybin and cucurbitacin analogs over quiescent and active stellate cells. In other words, compounds show higher anti-proliferative activity on active cells than on quiescent cells.

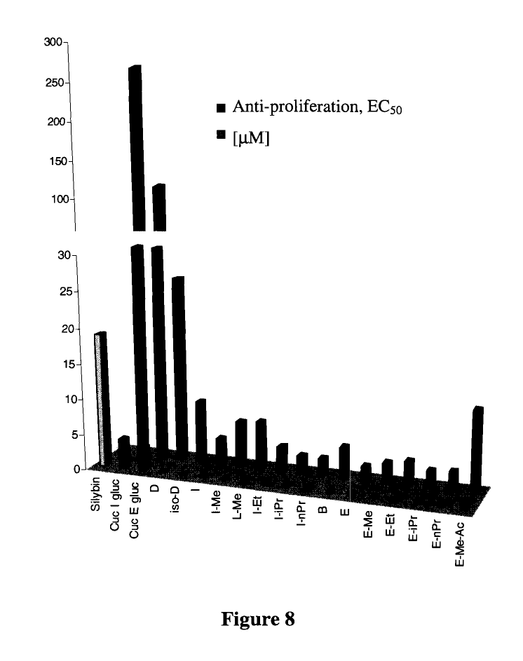

Compound toxicity was well characterized in experiment 3 (Table 8). The comparison between IC₅₀ and EC₅₀ on HSC-T6 cells (FIG. 8) show that much larger (T/A is 8-to-344) amount is required from the compounds to kill the cells than the active concentration.

Experimental conditions and calculation of activity suggested by Zhang regarding HSC-T6 cells proliferation with PDGF or serum were not reliable. We needed to change the cell concentration and FBS concentration in order to evaluate the antiproliferation effect of compounds due to the high cell confluency created by initial conditions. Furthermore, Zhang suggested that the inhibition would be calculated from the sample-to-positive control live cells ratio. We modified this approach. Our assay included a second control (ZC) supplemented with no serum intended to measure cucurbitacin anti-proliferation effect relative to the number of cells generated during challenge instead of the total number of cells present in each well.

Correlation Between Various Activities

A strong relationship was found between the cytotoxic activity of cucurbitacin and their ability to protect cells against CCl₄-induced toxicity, IC₅₀=5.058EC₅₀-0.517 (n=10, r=0.975). Hence, the cytotoxicity and cytoprotection mechanism may interfere at some point at cellular level. Cucurbitacin cytotoxicity on HepG2 cells does not show correlation with the cytotoxicity measured on HeLa cells (presented in chapter 3), and no correlation was found between their IC₅₀ and EC₅₀ values on HSC-T6 cells. The differential cytotoxicity of cucurbitacin on HeLa cells vs. HepG2 cells and non-correlation found on HSC-T6 suggest that there is no mechanistic interference at cellular level between the two bioactivities. Correlations between various bioactivities were investigated earlier by similar means.

Antioxidant Activity of Cucurbitacin

Oxidative processes appear to be of fundamental importance in the pathogenesis of cell damage in liver. Lipid peroxidation is a prominent phenomenon in several types of chemically induced hepatic injury, mostly due to the effect of free radicals, often produced via electrophile generation by cytochrome P450 isozyme superfamily metabolism. Recent studies have also drawn attention to a potentially important contribution of non-parenchymal liver cells and neutrophils via generation of reactive oxygen species in the pathogenesis of certain types of drug-induced liver injury.

Anti-Inflammatory Activity of Cucurbitacin

Cucurbitacin glycosides, nor-cucurbitacin glycosides, elaterium, cucurbitacin D, I, and E anti-inflammatory activity have been studied previously. In animal studies they inhibited induced edema, reduced vascular permeability, and the production of prostaglandin E₂. In contrast to cucurbitacin B, cucurbitacin D enhanced capillary permeability without any histamine releasing activity. The improved permeability was associated with a persistent fall in blood pressure and the accumulation of fluid in thoracic and abdominal cavities in mice. In vitro, cucurbitacin inhibited arachidonic acid release from neutrophils, suppressed the biosynthesis of eicosanoids in human leukocytes, and inhibited integrin-mediated cell adhesion of leukocytes by disrupting the cytoskeleton. The hyaluronidase enzyme plays an important role in inflammation because it depolymerizes hyaluronic acid in the connective tissue leading to the spread of chemotactic factors. Cucurbitacin B did not demonstrate anti-hyaluronidase activity.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A compound of the formula: 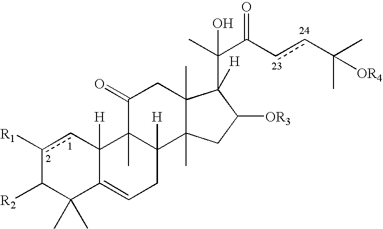
wherein:
R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo;
R₂ is —H, —OH, ═O, or (C₁-C₁₂)carboxylic acid;
R₃ is —H, (C₁-C₁₂)acyl, (C₁-C₁₂)alkyl, (C₁-C₁₂)alkene, or (C₁-C₁₂)carboxylic acid; and
R₄ is —H, (C₁-C₁₂)acyl, (C₁-C₁₂)alkyl, (C₁-C₁₂)alkene, or (C₁-C₁₂)carboxylic acid.

2. The compound of claim 1, wherein R₁ is selected from the group comprising β-D-glucopyranose, ═O, OH, methoxide, ethoxide, isopropoxide, and propoxide.

3. The compound of claim 1, wherein R₂ is selected from the group comprising ═O and OH.

4. The compound of claim 1, wherein R₃ is selected from the group comprising H and acetyl.

5. The compound of claim 1, wherein R₄ is selected from the group comprising H and acetyl.

6. The compound of claim 1, wherein carbon 1 and carbon 2 are bonded with a single bond.

7. The compound of claim 1, wherein carbon 1 and carbon 2 are bonded with a double bond.

8. The compound of claim 1, wherein carbon 23 and carbon 24 are bonded with a single bond.

9. The compound of claim 1, wherein carbon 23 and carbon 24 are bonded with a double bond.

10. The compound of claim 1, wherein carbon 1 and carbon 2 are bonded with a double bond and wherein carbon 23 and carbon 24 are bonded with a double bond.

11. A compound of the formula: 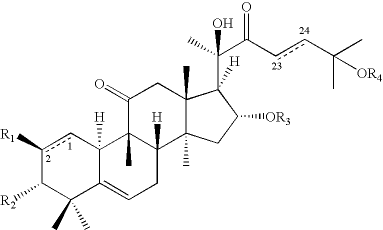
wherein
R₁ is —H, —OH, ═O, (C₁-C₁₂)alkyl, (C₁-C₁₂)alkoxide, (C₄-C₇)sugar, (C₁-C₁₂)acyl, (C₁-C₁₂alkene, (C₁-C₁₂)carboxylic acid, or halo;
R₂ is —H, —OH, ═O, or (C₁-C₁₂)carboxylic acid;
R₃ is —H, (C₁-C₁₂)acyl, (C₁-C₁₂)alkyl, (C₁-C₁₂)alkene, or (C₁-C₁₂)carboxylic acid; and
R₄ is —H, (C₁-C₁₂)acyl, (C₁-C₁₂)alkyl, (C₁-C₁₂)alkene, or (C₁-C₁₂)carboxylic acid.

12. The compound of claim 11, wherein R₁ is selected from the group comprising β-D-glucopyranose, ═O, OH, methoxide, ethoxide, isopropoxide, and propoxide.

13. The compound of claim 11, wherein R₂ is selected from the group comprising ═O and OH.

14. The compound of claim 11, wherein R₃ is selected from the group comprising H and acetyl.

15. The compound of claim 11, wherein R₄ is selected from the group comprising H and acetyl.

16. The compound of claim 11, wherein carbon 1 and carbon 2 are bonded with a double bond and wherein carbon 23 and carbon 24 are bonded with a single bond.

17. The compound of claim 11, wherein carbon 23 and carbon 24 are bonded with a double bond and wherein carbon 1 and carbon 2 are bonded with a single bond.

18. The compound of claim 11, wherein carbon 1 and carbon 2 are bonded with a double bond and wherein carbon 23 and carbon 24 are bonded with a double bond.

19. A compound of the formula: 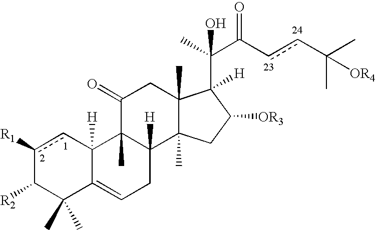
wherein
R₁ is —H, —OH, ═O, (C₁-C₁₂)alkyl, (C₁-C₁₂)alkoxide, (C₄-C₇)sugar, (C₁-C₁₂)acyl, (C₁-C₁₂)alkene, (C₁-C₁₂)carboxylic acid, or halo;
R₂ is —H, —OH, ═O, or (C₁-C₁₂)carboxylic acid;
R₃ is —H, (C₁-C₁₂)acyl, (C₁-C₁₂)alkyl, (C₁-C₁₂)alkene, or (C₁-C₁₂)carboxylic acid;
R₄ is —H, (C₁-C₁₂)acyl, (C₁-C₁₂)alkyl, (C₁-C₁₂)alkene, or (C₁-C₁₂)carboxylic acid;
carbon 1 and carbon 2 are bonded with a double bond, carbon 23 and carbon 24 are bonded with a double bond, or both carbon 1 and carbon 2 and carbon 23 and carbon 24 are bonded with a double bond.

20. The compound of claim 19, wherein R₁ is selected from the group comprising β-D-glucopyranose, ═O, OH, methoxide, ethoxide, isopropoxide, and propoxide; wherein R₂ is selected from the group comprising ═O and OH; wherein R₃ is selected from the group comprising H and acetyl; and wherein R₄ is selected from the group comprising H and acetyl.
